(19) United States
(12) Patent Application Publication
(10) Pub. No.: US 2023/0092490 A1
(43) Pub. Date: Mar. 23, 2023

(54) PHARMACEUTICAL COMPOSITIONS COMPRISING DASATINIB ANHYDROUS AND USES THEREOF

(71) Applicant: ZENTIVA K.S., Praha 10 (CZ)

(72) Inventors: Gregor SEDMAK, Praha 10 (CZ); Martin NOVAK, Praha (CZ); Ales BARTUNEK, Praha 10 (CZ); Jiri HOFMANN, Chocerady (CZ)

(73) Assignee: ZENTIVA K.S., Praha 10 (CZ)

(21) Appl. No.: 17/909,307

(22) PCT Filed: Mar. 5, 2021
(86) PCT No.: PCT/EP2021/055650

§ 371 Date: Sep. 2, 2022

(30) Foreign Application Priority Data
Mar. 6, 2020 (GB) 2003316.3

(51) Int. Cl. A61K 31/506; A61K 9/20; A61K 31/4439; A61K 45/06

(52) U.S. Cl. CPC A61K 31/506; A61K 9/2054; A61K 31/4439; A61K 45/06

(57) ABSTRACT

Pharmaceutical compositions comprising dasatinib anhydrous provide an improved, pH-independent dissolution profile compared with pharmaceutical compositions comprising dasatinib monohydrate. Thus, pharmaceutical compositions comprising dasatinib anhydrous can be used in the treatment of chronic myelogenous leukaemia (CML) and/or Philadelphia chromosome positive (Ph+) acute lymphoblastic leukaemia (ALL), especially in subjects having an increased gastric pH and/or in subjects being co-administered with a gastric acid reducing agent.

The present invention relates to pharmaceutical compositions comprising dasatinib. More specifically, this invention relates to pharmaceutical compositions comprising dasatinib anhydrous, together with its uses in the treatment of chronic myelogenous leukaemia (CML) and/or Philadelphia chromosome positive (Ph+) acute lymphoblastic leukaemia (ALL). This invention also relates to combined pharmaceutical preparations comprising dasatinib anhydrous and a gastric acid reducing agent.

### BACKGROUND

The compound dasatinib with the chemical formula N-(2-chloro-6-methylphenyl)-2-[[6-[4-(2-hydroxyethyl)-1-piperazinyl]-2-methyl-4-pyrimidinyl]amino]-5-thiazolecarboxamide is represented by the structure (I) below:

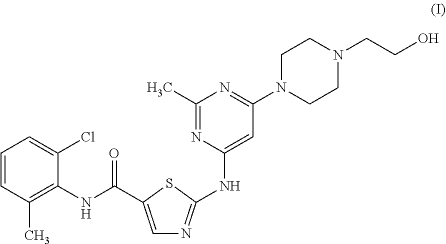 [SMILES: Cc1nc(Nc2ncc(C(=O)Nc3c(C)cccc3Cl)s2)cc(N2CCN(CCO)CC2)n1]

Dasatinib is available in the form of dasatinib monohydrate, also referred to herein as ‘DM’, as well as dasatinib anhydrous, also referred to herein as ‘DA’. Compositions and manufacturing methods for dasatinib monohydrate have been described in EP1885339. Methods of manufacturing dasatinib anhydrous have been described in WO2013065063A1. General processes for preparing aminothiazole-aromatic amides are described in WO 2005/077945 A2.

The expressions ‘dasatinib anhydrous’, ‘anhydrous dasatinib’ and ‘anhydrous form of dasatinib’ may be used interchangeably.

Dasatinib is an inhibitor of the BCR-ABL kinase and SRC family kinases along with a number of other selected oncogenic kinases including c-KIT, ephrin (EPH) receptor kinases, and PDGFβ receptor. Dasatinib is commonly used in the treatment of adults with chronic, accelerated or blast phase chronic myeloid leukaemia (CML) with resistance or intolerance to prior therapy including imatinib mesylate, and also for the treatment of adults with Philadelphia chromosome positive (Ph+) acute lymphoblastic leukaemia (ALL) and lymphoid blast CML with resistance or intolerance to prior therapy.

Dasatinib monohydrate is commercially available under the brand name SPRYCEL®, also referred to herein as the reference product ‘RP’. SPRYCEL® has been approved and is marketed in the United States and in Europe, as tablets containing 20, 50, 70, 80, 100 and 140 mg dasatinib monohydrate.

However, dasatinib monohydrate has the following disadvantages:

First, dasatinib monohydrate is characterized as a low solubility/high permeability (BCS II) compound according to the Biopharmaceutics Classification System. The solubility of dasatinib monohydrate is 8 μg/mL at 24° C. The oral bioavailability of dasatinib monohydrate is low with values ranging from 14% to 34%. Peak concentration is reached usually between 1-2 hrs. Therefore, the low solubility of dasatinib monohydrate limits the absorption of dasatinib monohydrate from the gastrointestinal tract which in turn reduces oral bioavailability of dasatinib monohydrate.

Second, dasatinib monohydrate exhibits pH-dependent thermodynamic (equilibrium) solubility (18.4 mg/ml at pH 2.6 to 0.008 mg/ml at pH 6.0). Consequently, the pH-dependent solubility and in turn the bioavailability of dasatinib monohydrate is affected by the natural variability of gastric pH. The normal gastric pH ranges from pH 1.5 to 3.3. However, the exact pH can vary among individual patients (interpatient variability) as well as for the same patient (intrapatient variability) depending, e.g., if the patient is in the fed or the fasted state. Therefore, the pH-dependent thermodynamic (equilibrium) solubility of dasatinib monohydrate results in a different bioavailability of dasatinib monohydrate.

Third, the administration of drugs regulating the gastric pH severely affects the solubility and in turn the bioavailability of dasatinib monohydrate. For example, proton pump inhibitors, histamine-2 (H2) antagonists and antacids may increase the gastric pH. Proton pump inhibitors and histamine-2 (H2) antagonists may act over a long period of time, i.e. in the range of 3 days to 7 days, e.g. 5 days, whereas, antacids may act for a short period of time, i.e. in the range of 1 to 5 hours, e.g. 2 hours. The co-administration of dasatinib monohydrate with antacids results in a reduction in bioavailability of dasatinib. Specifically, co-administration with aluminium hydroxide or magnesium hydroxide results in a 55% reduction of the area under the curve (AUC) for dasatinib monohydrate, and a 58% reduction in Cₘₐₓ. Bioavailability of dasatinib monohydrate is decreased when administered 10 hours following the H2 antagonist famotidine with AUC and Cₘₐₓ being reduced by 61% and 63%, respectively. It has also been shown that the administration of a single 100 mg dose of dasatinib monohydrate 22 hours following a 4-day 40-mg omeprazole dose at steady state reduced the AUC of dasatinib monohydrate by 43% and the Cₘₐₓ of dasatinib by 42% (SPRYCEL® summary of product characteristics, EMA). Thus, the co-administration of dasatinib and a H2 blocker (e.g. famotidine), proton pump inhibitor (e.g. omeprazole), or an antacid (e.g. aluminium hydroxide or magnesium hydroxide) may reduce the bioavailability to dasatinib.

A warning to this extent has been provided in the package information leaflet for dasatinib monohydrate sold under the brand name SPRYCEL® by the United States Food and Drug Administration (the “USFDA”) and by the European Medicines Agency (the “EMA”, see SmPC). In particular, the USFDA advises: (i) that co-administration of dasatinib monohydrate with a gastric acid reducing agent may decrease the concentrations of dasatinib; (ii) that decreased dasatinib concentrations may reduce efficacy; (iii) not to administer H2 antagonists or proton pump inhibitors with dasatinib monohydrate; (iv) to administer the antacid at least 2 hours prior to or 2 hours after the dose of dasatinib monohydrate; and (v) to avoid simultaneous administration of dasatinib monohydrate with antacids.

Thus, there is a need to provide an improved pharmaceutical composition comprising dasatinib that is independent of the effect of pH.

### SUMMARY OF INVENTION

According to the present invention the aforementioned problems have been solved by providing a pharmaceutical composition comprising dasatinib anhydrous.

The present invention further provides a pharmaceutical composition comprising dasatinib anhydrous for use in the treatment of chronic myelogenous leukaemia (CML) and/or Philadelphia chromosome positive (Ph+) acute lymphoblastic leukaemia (ALL).

In addition, the present invention relates to a combined pharmaceutical preparation comprising dasatinib anhydrous and a gastric acid reducing agent.

Further preferred or advantageous features of the invention are set out in the claims, infra.

### FIGURES

FIG. 1 shows the dissolution at pH 3.0 of dasatinib anhydrous (DA1) compared to dasatinib monohydrate (RP) (900 ml citrate buffer, 37° C., paddles at 60 RPM for 45 min, 150 RPM for final 15 min, t=50 minutes, every 5 minutes).

FIG. 2 shows the dissolution at pH 4.5 of dasatinib anhydrous (DA1) compared to dasatinib monohydrate (RP) (900 ml acetate buffer, 37° C., paddles at 75 RPM for 45 min, 150 RPM for final 15 min, t=50 minutes, every 5 minutes).

FIG. 3 shows the in-vivo release profile of dasatinib anhydrous in DA1 compared to dasatinib monohydrate (RP) in exemplary subject A.

FIG. 4 shows the in-vivo release profile of dasatinib anhydrous in DA1 compared to dasatinib monohydrate (RP) in exemplary subject B.

FIG. 5 shows the in-vivo release profile of dasatinib anhydrous in DA1 compared to dasatinib monohydrate (RP) in exemplary subject C.

FIG. 6 shows the in-vivo release profile of dasatinib anhydrous in DA1 compared to dasatinib monohydrate (DM) in exemplary subject D.

### DETAILED DESCRIPTION

According to the present invention, there is provided a pharmaceutical composition comprising dasatinib anhydrous. Preferably, the pharmaceutical composition comprises a pharmaceutically effective dose of dasatinib anhydrous. Advantageously, the pharmaceutical composition comprising dasatinib anhydrous provides a dosage form bioequivalent to dasatinib monohydrate but with less active pharmaceutical ingredient (“API”). Preferably, the pharmaceutical composition comprises about 20% to about 25% less API, more preferably from about 20% to about 23% less API, most preferably about 21% less API. Using less API reduces adverse effects which in turn further improves patient adherence, thus providing an improved pharmaceutical composition for use in the treatment of CML and/or Ph+ALL. Moreover, the pharmaceutical composition comprising dasatinib anhydrous provides an improved dissolution profile compared with a pharmaceutical composition comprising dasatinib monohydrate across different pH levels. Thus, the pharmaceutical composition of the invention is for co-administration with a gastric acid reducing agent. Additionally, the reduction of API when using dasatinib anhydrous over dasatinib monohydrate results in a significant reduction of treatment costs and potential side effects.

Optionally, the pharmaceutical composition comprising dasatinib anhydrous comprises about 15 mg to about 140 mg dasatinib anhydrous. The term “about”, when used to specify the amount of an API or any other components in pharmaceutical compositions of this invention, preferably means that the pharmaceutical composition contains the amount specified to ±10 mg, more preferably means that the pharmaceutical composition contains the amount specified to ±5 mg. Thus, “about 15 mg to about 140 mg dasatinib anhydrous” includes 5 mg to 150 mg dasatinib anhydrous, for example, 10 mg to 145 mg dasatinib anhydrous. Preferably, the pharmaceutical composition comprises about 40 mg to about 130 mg dasatinib anhydrous, more preferably about 80 mg to about 130 mg dasatinib anhydrous, most preferably about 100 mg to about 120 mg dasatinib anhydrous. Optionally, the pharmaceutical composition comprises about 110 mg dasatinib anhydrous.

For example, the pharmaceutical composition comprising dasatinib anhydrous may comprise at least about 30 mg, at least about 31 mg, at least about 32 mg, at least about 33 mg, at least about 34 mg, at least about 35 mg, at least about 36 mg, at least about 37 mg, at least about 38 mg, at least about 39 mg, at least about 40 mg of dasatinib anhydrous. The pharmaceutical composition comprising dasatinib anhydrous may comprise no more than about 50 mg, no more than about 49 mg, no more than about 48 mg, no more than about 47 mg, no more than about 46 mg, no more than about 45 mg, no more than about 44 mg, no more than about 43 mg, no more than about 42 mg, no more than about 41 mg, no more than about 40 mg of dasatinib anhydrous. The pharmaceutical composition comprising dasatinib anhydrous may comprise about 30 mg to about 50 mg, about 31 mg to about 49 mg, about 32 mg to about 48 mg, about 33 mg to about 47 mg, about 34 mg to about 46 mg, about 35 mg to about 45 mg, about 36 mg to about 44 mg, about 37 mg to about 43 mg, about 38 mg to about 42 mg, about 39 mg to about 41 mg dasatinib anhydrous. The pharmaceutical composition comprising dasatinib anhydrous may comprise any range from the given endpoints. The pharmaceutical composition comprising dasatinib anhydrous may provide a dosage form bioequivalent to dasatinib monohydrate but with less active API.

For example, the pharmaceutical composition comprising dasatinib anhydrous may comprise at least about 60 mg, at least about 62 mg, at least about 64 mg, at least about 66 mg, at least about 68 mg, at least about 70 mg, at least about 72 mg, at least about 74 mg, at least about 76 mg, at least about 78 mg, at least about 80 mg of dasatinib anhydrous. The pharmaceutical composition comprising dasatinib anhydrous may comprise no more than about 100 mg, no more than about 98 mg, no more than about 96 mg, no more than about 94 mg, no more than about 92 mg, no more than about 90 mg, no more than about 88 mg, no more than about 86 mg, no more than about 84 mg, no more than about 82 mg, no more than about 80 mg of dasatinib anhydrous. The pharmaceutical composition comprising dasatinib anhydrous may comprise about 60 mg to about 100 mg, about 62 mg to about 98 mg, about 64 mg to about 96 mg, about 66 mg to about 94 mg, about 68 mg to about 92 mg, about 70 mg to about 90 mg, about 72 mg to about 88 mg, about 74 mg to about 86 mg, about 76 mg to about 84 mg, about 78 mg to about 82 mg dasatinib anhydrous. The pharmaceutical composition comprising dasatinib anhydrous may comprise any range from the given endpoints. The pharmaceutical composition comprising dasatinib anhydrous may provide a dosage form bioequivalent to dasatinib monohydrate but with less active API.

Optionally, the pharmaceutical composition comprises 110.6 mg dasatinib anhydrous; 149.3 mg lactose monohydrate; 149.3 mg microcrystalline cellulose; 13.3 mg hydroxypropylcellulose; 17.7 mg croscarmellose sodium; 2.2 mg magnesium stearate; 7.9 mg hypromellose; 0.8 mg propylene glycol; and 2.8 mg titanium dioxide. Advantageously, this pharmaceutical composition comprising 110.6 mg dasatinib is bioequivalent to a pharmaceutical composition comprising 140 mg dasatinib monohydrate.

Optionally, the pharmaceutical composition comprising dasatinib anhydrous is for oral administration. Preferably, the pharmaceutical composition comprising dasatinib anhydrous is in tablet form. The tablet form may be a coated tablet, for example, a film-coated tablet.

According to the present invention, there is also provided a pharmaceutical composition comprising dasatinib anhydrous for use in the treatment of chronic myelogenous leukaemia (CML) and/or Philadelphia chromosome positive (Ph+) acute lymphoblastic leukaemia (ALL). The subject may be a human subject; male or female. The subject may be above 18 years, above 25 years, above 40 years, above 50 years, above 60 years, above 70 years, above 80 years. The terms “subject” and “patient” may be used interchangeably. The use in the treatment of chronic myelogenous leukaemia (CML) and/or acute lymphoblastic leukaemia (ALL) such as Philadelphia chromosome positive (Ph+) acute lymphoblastic leukaemia (ALL) includes the reduction, prevention and/or delay of chronic myelogenous leukaemia (CML) and/or Philadelphia chromosome positive (Ph+) acute lymphoblastic leukaemia (ALL). Advantageously, the pharmaceutical composition comprising dasatinib anhydrous for use in the treatment of chronic myelogenous leukaemia (CML) and/or Philadelphia chromosome positive (Ph+) acute lymphoblastic leukaemia (ALL) provides a dosage form bioequivalent to dasatinib monohydrate but with less API. Preferably, the pharmaceutical composition comprises between about 20% to about 25% less API than the reference product SPRYCEL®. In particularly preferred embodiments, the pharmaceutical composition comprises between about 20% to about to about 23% less API than the reference product SPRYCEL®), and most preferably about 21% less API than the reference product SPRYCEL®. For example, SPRYCEL® tablets containing dosages of 20, 50, 70, 80, 100 and 140 mg dasatinib monohydrate are commercially available in the United States and Europe. Hence, in a preferred embodiment, the dosage forms of this invention may comprise dasatinib anhydrous in reduced amount that is between about 20% to about 25% less than the dosages of SPRYCEL®, and even more preferably a dosage of dasatinib anhydrous that is about 21% less than the foregoing dosages in SPRYCEL. Hence, in particularly preferred embodiments, a pharmaceutical composition of the present invention may contain an amount (or range of amounts) of dasatinib anhydrous as indicated in the following table and corresponding to each commercially available dose for SPRYCEL® (dasatinib monohydrate) as indicated in Table 1 below.

TABLE 1
Compositions comprising dasatinib monohydrate and dasatinib anhydrous.

| SPRYCEL ® Dosage | Dasatinib Anhydrous | |
|---|---|---|
| Monohydrate) | (Corresponding Dosages) | |
| (Dasatinib | (20-25% Reduction) | (21% Reduction) |
| 20 mg | 15.0-16.0 mg | 15.8 mg |
| 50 mg | 37.5-40 mg | 39.5 mg |
| 70 mg | 52.5-56 mg | 55.3 mg |
| 80 mg | 60-64 mg | 63.2 mg |
| 100 mg | 75-80 mg | 79 mg |
| 140 mg | 105-112 mg | 110.6 mg |

The pharmaceutical composition comprising dasatinib anhydrous is particularly suited for use in the treatment of CML and/or Ph+ALL in a subject having an increased gastric pH. An increased gastric pH is a gastric pH which is above the normal gastric pH. The skilled person readily knows how to determine the gastric pH of a subject. For example, gastric pH can be measured by catheter-based monitoring using a pH catheter inserted transnasally into the stomach. In particular, the skilled person is readily able to determine a normal gastric pH, i.e. a gastric pH of 1.5 to 3.3. The skilled person is also readily able to determine an increased gastric pH, i.e. an above-normal gastric pH e.g. a gastric pH greater than 3.3, such as a pH selected from pH 3.4, pH 3.5, pH 3.6, pH 3.7, pH 3.8, pH 3.9, pH 4.0, pH 4.5, pH 5.0, pH 5.5, pH 6.0, pH 6.5, pH 7.0.

Advantageously, a pharmaceutical composition comprising dasatinib anhydrous provides an improved treatment in subjects having an increased gastric pH. While the pharmaceutical compositions and uses of this invention are not limited to any particular theory or mechanism of action, these and other advantages of the invention are believed to be due, at least in part, to significant differences in the dissolution rate (kinetics) of dasatinib monohydrate and dasatinib anhydrous. In particular, the dissolution rates of dasatinib monohydrate and dasatinib anhydrous are similar at normal gastric pH, whereas dasatinib anhydrous has higher dissolution rates at increased gastric pH compared with dasatinib monohydrate. Accordingly dasatinib anhydrous has improved bioavailability, in particular in subjects having an increased gastric pH. Thus, the pharmaceutical composition comprising dasatinib anhydrous provides an improved treatment in subjects having an increased gastric pH.

Optionally, the pharmaceutical composition comprising dasatinib anhydrous is for use in the treatment of CML and/or Ph+ALL in a subject having a gastric pH of about pH 3 to about pH 8; about pH 3.5 to about pH 7.5; about pH 4 to about pH 7; about pH 4.5 to about pH 6.5.

Optionally, the pharmaceutical composition comprising dasatinib anhydrous is co-administered with a gastric acid reducing agent. The pharmaceutical composition comprising dasatinib anhydrous is also for use in the treatment of CML and/or Ph+ALL in a subject co-administered with a gastric acid reducing agent. The skilled person readily understands that co-administration with a gastric acid reducing agent means that dasatinib anhydrous is administered at any point during the efficacy period of the gastric reducing agent. The co-administration of dasatinib anhydrous and the gastric reducing agent may be concomitant, simultaneous or sequential. For example, a pharmaceutical composition comprising dasatinib anhydrous may be co-administered 22 hours following a 4-day 40 mg omeprazole dose. The gastric acid reducing agent may have the capacity to decrease gastric acid to such an extent that the gastric pH is higher than pH 5 (strong gastric acid reducing agent), higher than pH 4 (medium gastric acid reducing agent) or higher than pH 3.3 (weak gastric acid reducing agent). The gastric acid reducing agent may be a proton pump inhibitor, histamine-2 (H2) antagonist and/or an antacid. Advantageously, the pharmaceutical composition comprising dasatinib anhydrous for use in a subject co-administered with a gastric acid reducing agent provides improved bioavailability which is independent of the co-administration with gastric acid reducing agents. Increased bioavailability can be measured by the AUC(0-t) and Cₘₐₓ of dasatinib anhydrous compared with dasatinib monohydrate.

Optionally, the pharmaceutical composition comprising dasatinib anhydrous is co-administered with a proton pump inhibitor. Subjects being treated for CML and/or ALL/Ph+ALL often also require administration of a proton pump inhibitor. For example, the proton pump inhibitors may be administered five days prior to administration of dasatinib anhydrous. The term “proton pump inhibitor” includes but is not limited to proton pump inhibitors forming the state of the art. Preferably, the proton pump inhibitor is selected from one or more of omeprazole, esomeprazole, lansoprazole, dexlansoprazole, pantoprazole and rabeprazole. Advantageously, the pharmaceutical composition comprising dasatinib anhydrous for use in a subject co-administered with a proton pump inhibitor provides improved bioavailability. This enables patients to receive a clinically relevant dasatinib dose despite being co-administered with a proton pump inhibitor. The pharmaceutical composition comprising dasatinib anhydrous may be for use in the treatment of CML and Ph+ALL in a subject, wherein the subject is co-administered with omeprazole.

Optionally, the pharmaceutical composition comprising dasatinib anhydrous is co-administered with a histamine-2 (H2) antagonist. Subjects being treated for CML and/or Ph+ALL often also require administration of a histamine-2 (H2) antagonist. For example, the histamine-2 (H2) antagonist may be administered five days prior to administration dasatinib anhydrous. The term “histamine-2 (H2) antagonist” is not limited to histamine-2 (H2) antagonists forming the state of the art. Preferably, the histamine-2 (H2) antagonist is selected from one or more of famotidine, cimetidine, ranitidine, nizatidine, roxatidine and lafutidine. Advantageously, the pharmaceutical composition comprising dasatinib anhydrous for use in a subject co-administered with a histamine-2 (H2) antagonist provides improved bioavailability. This enables patients to receive a clinically relevant dasatinib dose despite being co-administered with a histamine-2 (H2) antagonist.

Optionally, the pharmaceutical composition comprising dasatinib anhydrous is co-administered with an antacid. The skilled person readily knows that antacids are medicines that counteract the stomach acid to relieve the symptoms of gastroesophageal reflux disease, heartburn or indigestion. Subjects being treated for CML and/or Ph+ALL often also require administration of an antacid. For example, the gastric acid reducing agent may be administered less than 2 hours prior to or 2 hours after the dose of dasatinib anhydrous. The term “antacid” includes but is not limited to antacids forming the state of the art. Preferably, the antacid is selected from aluminium hydroxide, calcium carbonate and/or sodium bicarbonate. Advantageously, the pharmaceutical composition comprising dasatinib anhydrous for use in a subject co-administered with an antacid provides improved bioavailability. This enables patients to receive a clinically relevant dasatinib dose despite being co-administered with an antacid.

Optionally, the pharmaceutical composition comprising dasatinib anhydrous is for use in the treatment of CML and/or Ph+ALL in a subject having achlorhydria or hypochlorhydria. The skilled person readily knows that achlorhydria and hypochlorhydria is the absence or reduction of production of hydrochloric acid in gastric secretions. The skilled person also readily knows how to identify a person having achlorhydria or hypochlorhydria, e.g. by catheter-based monitoring using a pH catheter inserted transnasally into the stomach. Achlorhydria and hypochlorhydria may result in an increased gastric pH. Advantageously, the pharmaceutical composition comprising dasatinib anhydrous provides an improvement over dasatinib monohydrate in patients having achlorhydria or hypochlorhydria. In bioequivalence studies performed with dasatinib monohydrate, 3.5% to 7.5% of subjects exhibit atypical pharmacokinetic profiles likely connected to achlorhydria or hypochlorhydria, in which bioavailability of dasatinib monohydrate is reduced. A pharmaceutical composition comprising dasatinib anhydrous enables patients to receive a clinically relevant dasatinib dose despite having achlorhydria or hypochlorhydria.

Optionally, the pharmaceutical composition comprising dasatinib anhydrous is for use in the treatment of CML and/or Ph+ALL in a subject being 50 years.

Achlorhydria increases with age. For example, the prevalence of achlorhydria in healthy subjects is between 1% to 5%, which increases with age to 19% for subjects in their 50s and 69% for subjects in their 80s. This increase of achlorhydria prevalence with age is particularly problematic considering that the average age of diagnosis of CML is 64 years. Thus, the pharmaceutical composition comprising dasatinib anhydrous advantageously provides an improvement over dasatinib monohydrate in a subject 50 years.

According to the present invention, there is also provided a combined pharmaceutical preparation comprising dasatinib anhydrous and a gastric acid reducing agent. The gastric acid reducing agent may be a proton pump inhibitor, e.g. omeprazole, or a histamine-2 (H2) antagonist, e.g. famotidine, and/or an antacid, e.g. aluminium hydroxide, calcium carbonate and/or sodium bicarbonate. Preferably, the combined pharmaceutical preparation comprises dasatinib anhydrous and one or more of omeprazole, esomeprazole, lansoprazole, dexlansoprazole, pantoprazole and rabeprazole. Advantageously, the combined pharmaceutical preparation comprising dasatinib anhydrous and a gastric acid reducing agent provides an effective pharmaceutical preparation comprising dasatinib which is independent of the reduced pH resulting from the gastric acid reducing agent.

The present invention is now described in more detail by, but is not limited to, the following Examples.

### EXAMPLES

Sample Preparation

DA1 comprising 140 mg dasatinib anhydrous was compared with RP comprising 140 mg dasatinib monohydrate, as set out in Table 2 below.

DA1 differed from RP only in the API form, i.e. DA1 comprised dasatinib anhydrous whereas RP comprised dasatinib monohydrate. The amount of active ingredient (140 mg) was the same in DA1 and RP.

TABLE 2
Formulation comparison of RP comprising 140 mg dasatinib monohydrate
and DA1 comprising 140 mg dasatinib anhydrous.

| Pharmaceutical | | DA1 | RP |
|---|---|---|---|
| composition | Function | mg/tbl | mg/tbl |
| Dasatinib anhydrous | API | 140.00 | — |
| Dasatinib monohydrate | API | — | 140.00 |
| Lactose monohydrate | Filler | 189.00 | 189.00 |
| Microcrystalline cellulose | Filler | 189.00 | 189.00 |
| Hydroxypropylcellulose | Binder | 16.80 | 16.80 |
| Croscarmellose sodium | Disintegrant | 22.40 | 22.40 |
| Magnesium stearate | Lubricant | 2.80 | 2.80 |
| Hypromellose | Coating agent | 10.05 | 10.05 |
| Propylene glycol | Coating agent | 1.01 | 1.01 |
| Titanium dioxide | Coating agent | 3.49 | 3.49 |

DA1 was prepared by introducing dasatinib anhydrous, lactose monohydrate, microcrystalline cellulose, hydroxypropylcellulose and croscarmellose sodium into a suitable high shear mixer and premixing. The premix was granulated with purified water. The resulting granulate was dried in a fluid bed dryer and sieved through a 0.8 mm sieve. The sieved granulate was mixed with magnesium stearate and the resulting tableting mixture was compressed on a high speed rotary tableting machine. Subsequently, the tablets were coated with coating an agent dissolved/suspended in purified water.

Formulations comprising approximately 110 mg dasatinib anhydrous (DA2) were also prepared according to the above method and are provided for in Table 3 below.

TABLE 3
Pharmaceutical composition comprising 110 mg
dasatinib anhydrous (DA2)

| Pharmaceutical composition | Function | mg/tbl |
|---|---|---|
| dasatinib anhydrous | API | 110.6 |
| Lactose monohydrate | Filler | 149.3 |
| Microcrystalline cellulose | Filler | 149.3 |
| Hydroxypropylcellulose | Binder | 13.3 |
| Croscarmellose sodium | Disintegrant | 17.7 |
| Magnesium stearate | Lubricant | 2.2 |
| Hypromellose | Coating agent | 7.9 |
| Propylene glycol | Coating agent | 0.8 |
| Titanium dioxide | Coating agent | 2.8 |

### Example 1—In-Vitro Dissolution Assays

The dissolution of dasatinib anhydrous was compared with the dissolution of dasatinib monohydrate at different pH levels. More specifically, dissolution at pH 3 and pH 4.5 was tested. Dissolution measurements were performed using USP 2 paddle apparatus and 900 mL of buffer, pH 3.0 was prepared by using 50 mM citrate buffer and pH 4.5 was prepared using 50 mM acetate buffer. For pH 3.0, the stirring rate was 60 RPM for the first 45 minutes and then increased to 150 RPM for the last 15 minutes of the experiment. For pH 4.5, the stirring rate was 75 RPM for the first 45 minutes and then increased to 150 RPM for the last 15 minutes of the experiment. The dissolution assay was conducted at 37° C.

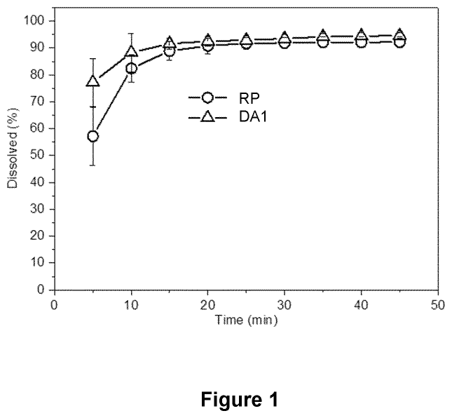

At pH 3 (i.e. normal gastric pH), the dissolution profile of DA1 (triangular symbol) did not differ significantly from that of RP (circular symbol). For example, about 90% of DA1 and about 90% of RP were entered solution at pH 3 after 30 minutes—see FIG. 1.

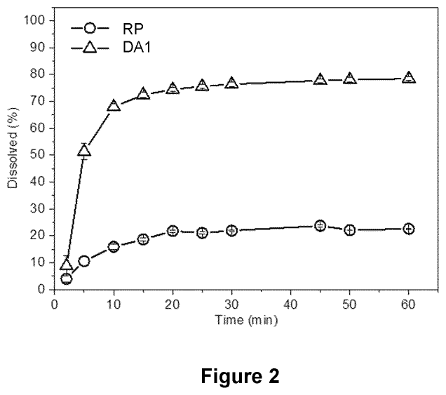

At pH 4.5 (i.e. above-normal gastric pH) the dissolution profile of DA1 (triangular symbol) differed significantly from the dissolution profile of RP (circular symbol). For example, about 75% of DA1 but only 20% of RP entered solution at pH 4.5 after 30 minutes. Similarly, about 75% of DA1 but only 20% of RP entered solution at pH 4.5 after 60 minutes—see FIG. 2.

These data demonstrate that a pharmaceutical composition comprising dasatinib anhydrous has an improved dissolution profile compared with dasatinib monohydrate across different pH levels. In particular, dasatinib anhydrous has greater dissolution compared with dasatinib monohydrate at above-normal gastric pH levels, for example at pH 4.5.

Summary of In-Vivo Studies

The Applicant conducted in-vivo studies with the formulations RP and DA1 of Table 2 and also with the formulations RP and DA2 of Table 3. These studies and their results are summarized in the following examples.

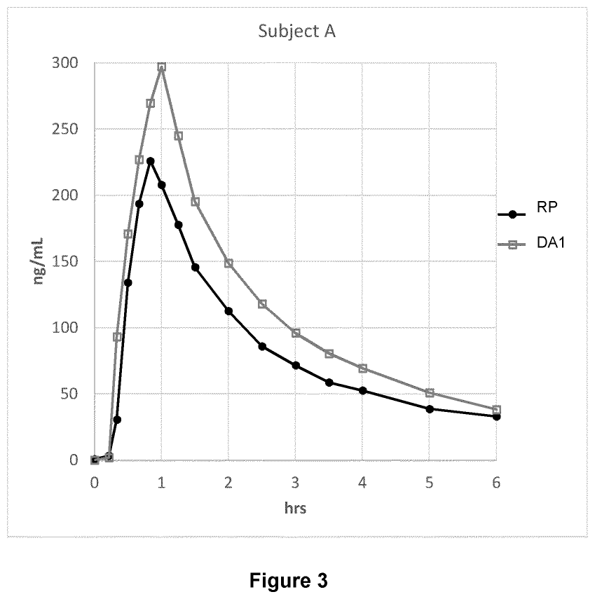

First, a preliminary bioequivalence study assessed in-vivo absorption of DA1 and RP. This bioequivalence study and its results are summarized in Example 2, below, and data are shown in FIG. 3 to FIG. 6 for four exemplary subjects A to D in that study.

Example 3, together with corresponding Table 4, presents additional results from the clinical study of Example 2, comparing the bioavailability of DA1 vs RP.

Example 4, together with corresponding Table 5, presents results from a clinical study that compared the bioavailability of DA1 vs RP, when each dosage form is co-administered with 40 mg omeprazole.

Example 5, together with Table 6, summarizes results from a fourth clinical study comparing the bioavailability of 110 mg dasatinib anhydrous (DA2) with RP.

### Example 2—Preliminary Bioequivalence Patient Comparison of DA1 vs. RP

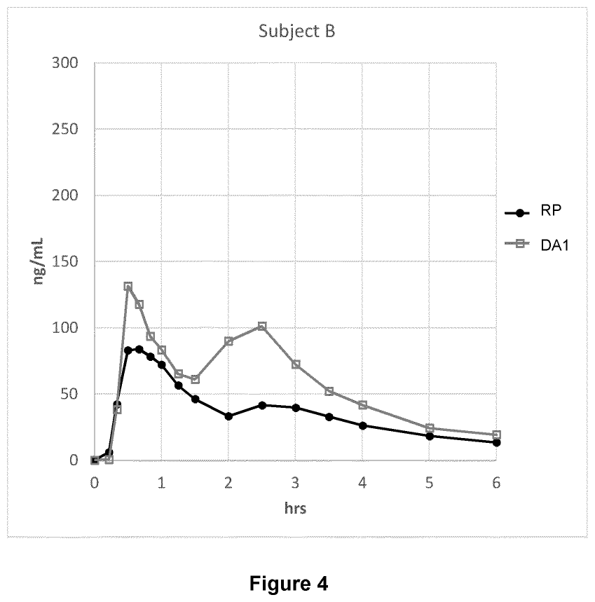

A preliminary bioequivalence study in eighty (80) healthy subjects was conducted to test the in-vivo absorption of RP and DA1. As shown for exemplary subjects A and B in FIG. 3 and FIG. 4, in-vivo absorption of dasatinib anhydrous from DA1 (white box symbol) was similar to dasatinib monohydrate from RP (black circle symbol).

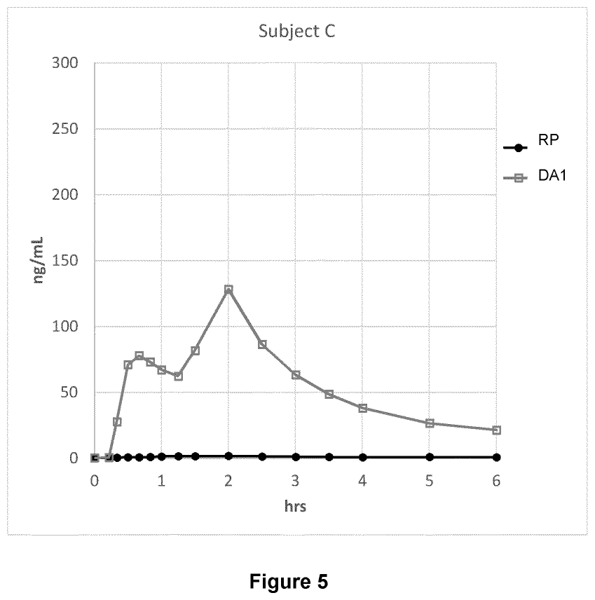

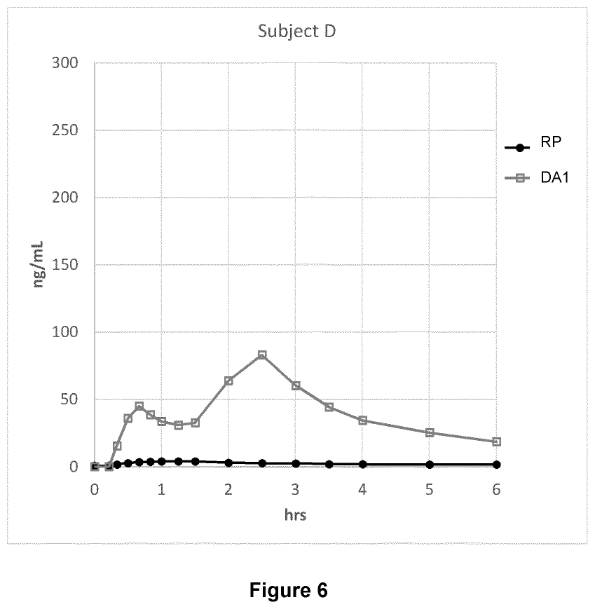

FIG. 5 and FIG. 6 demonstrate that the in-vivo absorption of dasatinib anhydrous from DA1 (white box symbol) was improved compared with dasatinib monohydrate from RP (black circle symbol) in exemplary subjects C and D, respectively.

For this sub-group, DA1 showed an absorption of dasatinib anhydrous similar to that of other exemplary subjects (i.e. subjects A and B), whereas the RP resulted in only very low absorption of dasatinib monohydrate, i.e. almost a zero amount of dasatinib, as depicted in FIG. 5 and FIG. 6.

These data demonstrate that kinetic solubility and absorption differed between dasatinib anhydrous and dasatinib monohydrate. Dasatinib anhydrous was absorbed in all subjects whereas dasatinib monohydrate was not—i.e. not in exemplary subjects C and D. This highlights the interpatient variability of bioavailability of dasatinib monohydrate, whereas dasatinib anhydrous shows consistent bioavailability across the total patient population.

### Example 3—Bioequivalence study of DA1 vs. RP

Additional data from the bioequivalence study of Example 2, which compared the bioequivalence of DA1 against the reference product (“RP”) are summarised in Table 4 below.

TABLE 4
Results of bioequivalence study of DA 140 mg
(DA1) vs. 140 mg DM (RP)

| | | 90% CONFIDENCE | |
|---|---|---|---|
| | | LIMITS (%) | |
| Parameter | RATIO T/R (%) | Lower | Upper |
| AUC(0-t) | 140.25 | 126.29 | 155.75 |
| Cₘₐₓ | 134.27 | 117.34 | 153.63 |

Table 4 above demonstrates that a pharmaceutical composition comprising dasatinib anhydrous 140 mg leads to an increase in bioavailability compared with dasatinib monohydrate 140 mg.

### Example 4—Co-Administration of DA1 and RP with Omeprazole

Thirty-six (36) healthy volunteers received dasatinib anhydrous and dasatinib monohydrate, each co-administered with 40 mg omeprazole at steady-state. DA1 and RP were administered within 12 hours of the last omeprazole dose.

A bioequivalence study comparing DA1 with RP was performed and results of the study are shown in Table 5.

TABLE 5
Dasatinib anhydrous 140 mg (DA1) vs. dasatinib monohydrate 140 mg
(RP), co-administered with omeprazole 40 mg.

| | | 90% CONFIDENCE | |
|---|---|---|---|
| | | LIMITS (%) | |
| Parameter | RATIO T/R (%) | Lower | Upper |
| AUC(0-t) | 487.76 | 391.97 | 606.97 |
| Cₘₐₓ | 557.69 | 408.38 | 761.58 |

Table 5 above demonstrates that a pharmaceutical composition comprising dasatinib anhydrous 140 mg leads to considerable increase in bioavailability of about 4 to 5-times compared with dasatinib monohydrate 140 mg after pre-treatment with 40 mg omeprazole.

### Example 5—Dose Finding

Due to the improved bioavailability of DA1 compared with RP, a new formulation (DA2) was prepared, in which the dose of dasatinib anhydrous was reduced by 21% from 140 mg (DA1) to approximately 110 mg (DA2). The bioequivalence of DA2 compared with RP was evaluated in forty (40) healthy volunteers. The results are summarized in Table 6, below.

The resulting product, comprising 110.60 mg DA (DA2), showed bioequivalence against the reference product comprising 140 mg DM (RP) as evidenced by the results shown in Table 6.

TABLE 6
Results of bioequivalence study of DA2 vs. RP.

| | | 90% CONFIDENCE | |
|---|---|---|---|
| | | LIMITS (%) | |
| Parameter | RATIO T/R (%) | Lower | Upper |
| AUC(0-t) | 99.36 | 89.39 | 110.43 |
| Cₘₐₓ | 100.39 | 86.83 | 116.06 |

1. A pharmaceutical composition comprising dasatinib anhydrous.

2. The pharmaceutical composition according to claim 1, comprising dasatinib anhydrous in an amount that is from about 20% to about 25% less than the amount of dasatinib monohydrate in a bioequivalent pharmaceutical composition comprising dasatinib monohydrate.

3. The pharmaceutical composition according to claim 1 or 2 comprising about 15 mg to about 140 mg dasatinib anhydrous, preferably about 40 mg to about 130 mg dasatinib anhydrous, more preferably about 80 mg to about 130 mg dasatinib anhydrous, most preferably about 100 mg to about 120 mg dasatinib anhydrous.

4. The pharmaceutical composition according to claim 1 or 2, comprising an amount of dasatinib anhydrous that is:
from about 15 mg to about 16 mg;
from about 37.5 mg to about 40 mg;
from about 52.5 to about 56 mg;
from about 60 to about 64 mg;
from about 75 to about 80 mg; or
from about 105 to about 112 mg.

5. The pharmaceutical composition according to claim 1 or 2, comprising about 15.8 mg, about 39 mg, about 55 mg, about 63 mg, about 79 mg or about 110 mg dasatinib anhydrous.

6. The pharmaceutical composition according to claim 5 comprising about 110 mg dasatinib anhydrous.

7. The pharmaceutical composition according to any preceding claim, wherein the pharmaceutical composition is for oral administration.

8. The pharmaceutical composition according to any preceding claim, wherein the pharmaceutical composition is in tablet form.

9. A pharmaceutical composition according to any preceding claim for use in the treatment of chronic myelogenous leukaemia (CML) and/or Philadelphia chromosome positive (Ph+) acute lymphoblastic leukaemia (ALL).

10. The pharmaceutical composition for use according to claim 9 in a subject having increased gastric pH.

11. The pharmaceutical composition for use according to claim 10, wherein the subject has an increased gastric pH of about pH 3 to about pH 7, preferably about pH 3.3 to about pH 5, more preferably about pH 3.5 to about pH 4.5.

12. The pharmaceutical composition for use according to claims 9 to 11, wherein the pharmaceutical composition is co-administered with a gastric acid reducing agent.

13. The pharmaceutical composition for use according to claim 12, wherein the gastric acid reducing agent is a proton pump inhibitor.

14. The pharmaceutical composition for use according to claim 13, wherein the proton pump inhibitor is selected from one or more of omeprazole, esomeprazole, lansoprazole, dexlansoprazole, pantoprazole and rabeprazole.

15. The pharmaceutical composition for use according to claim 12, wherein the gastric acid reducing agent is a histamine-2 (H2) antagonist.

16. The pharmaceutical composition for use according to claim 15, wherein the histamine-2 (H2) antagonist is selected from one or more of famotidine, cimetidine, ranitidine, nizatidine, roxatidine, or lafutidine.

17. The pharmaceutical composition for use according to claim 12, wherein the gastric acid reducing agent is an antacid.

18. The pharmaceutical composition for use according to claim 17, wherein the antacid is aluminium hydroxide, calcium carbonate and/or sodium bicarbonate.

19. The pharmaceutical composition for use according to claims 9 to 18 in a subject having achlorhydria or hypochlorhydria.

20. The pharmaceutical composition for use according to claims 9 to 19 in a subject being 50 years.

21. A combined pharmaceutical preparation comprising dasatinib anhydrous and a gastric acid reducing agent.

22. A combined pharmaceutical preparation according to claim 21 comprising dasatinib anhydrous and one or more of omeprazole, esomeprazole, lansoprazole, dexlansoprazole, pantoprazole and rabeprazole.
